(19) United States
(12) Patent Application Publication
(10) Pub. No.: US 2023/0351175 A1
(43) Pub. Date: Nov. 2, 2023

(54) METHOD FOR TRAINING MACHINE-LEARNING MODEL FOR INFERRING MOTION COORDINATION, APPARATUS FOR INFERRING MOTION COORDINATION USING MACHINE-LEARNING MODEL, AND STORAGE MEDIUM STORING INSTRUCTIONS TO PERFORM METHOD FOR TRAINING MACHINE-LEARNING MODEL FOR INFERRING MOTION COORDINATION

(71) Applicant: Research & Business Foundation SUNGKYUNKWAN UNIVERSITY, Suwon-si (KR)

(72) Inventors: Seung-Chan KIM, Suwon-si (KR); Hyejoo KIM, Suwon-si (KR); Hyerin KIM, Suwon-si (KR); Hyeon-Joo KIM, Suwon-si (KR)

(73) Assignee: Research & Business Foundation SUNGKYUNKWAN UNIVERSITY, Suwon-si (KR)

(21) Appl. No.: 18/130,992

(22) Filed: Apr. 5, 2023

(30) Foreign Application Priority Data
Apr. 5, 2022 (KR) 10-2022-0042379

(51) Int. Cl. G06N 3/08

(52) U.S. Cl. CPC G06N 3/08

(57) ABSTRACT

An artificial neural network model training method includes acquiring a plurality of motion data items including each motion data item for a plurality of parts of a moving body; calculating coordination between parts of the moving body based on correlation between the plurality of motion data items; and training an artificial neural network model using a training dataset including at least one motion data item among the plurality of motion data items as an input data item, and the coordination between the plurality of parts as a target variable.

### TECHNICAL FIELD

The present disclosure relates to an artificial neural network model training apparatus for inferring motion coordination, an artificial neural network model training method performed by the apparatus, and a motion coordination inferring apparatus using an artificial neural network model and a motion coordination inferring method performed by the apparatus.

This work was supported by Institute of Information & communications Technology Planning & Evaluation (IITP) grant funded by the Korea government(MSIT) (Project unique No.: 1711125814; Project No.: 2019-0-00050; R&D project: Artificial Intelligence Convergence Leading Project; and Research Project Title: Development of Health Behavior Monitoring, Diagnosis, and Prescription Technology Applying Natural Intelligence Emulation Artificial Intelligence Framework and Cognitive-based Convergence Research and Development Pipeline) and Korea Evaluation Institute of Industrial Technology (KEIT) that was funded by Ministry of Trade, Industry and Energy of Korea (MOTIE)(Project unique No.: 1415184394; Project No.: 20015188; R&D project: Artificial Intelligence Convergence Leading Project; and Research Project Title: Development of AI-based motion analysis coaching and visualization technology to improve the accuracy of user posture and enhance trainer coaching efficiency during 1:N non-face-to-face sports training).

### BACKGROUND ART

With the development of sensors and wearable technologies for measuring body movements, human activity recognition has been actively studied.

In general, in order to measure body balance and coordination between body parts in activities such as walking, a method of measuring correlation between motions by attaching a device including multiple sensors to the body is used. For example, in a walking activity, movement of the left and right arms is measured and compared to measure the left/right symmetry of the movement.

This method for measuring coordination/balance between body parts using multiple sensors has great significance in that it quantifies the characteristics of body movements, but has limitations and is not practical in that multiple sensors must be used.

### DISCLOSURE

### Technical Problem

According to an embodiment, there is provided an artificial neural network model training method and apparatus for training an artificial neural network model to infer coordination between body parts based on similarity between motion data items for a plurality of parts according to motion of a moving body.

Also, there is provided a motion coordination inferring method and apparatus for inferring coordination between body parts of a target moving body as an output of a trained artificial neural network model by inputting motion data items for a plurality of parts of a target moving body to the trained artificial neural network model.

It should be noted that objectives of the present disclosure are not limited to the above-described objects, and other objectives of the present disclosure will be apparent to those skilled in the art from the following descriptions.

### Technical Solution

In accordance with a first aspect of the present disclosure, there is provided an artificial neural network model training method performed by an artificial neural network model training apparatus for inferring motion coordination, the method comprises: acquiring a plurality of motion data items including each motion data item for a plurality of parts of a moving body; calculating coordination between parts of the moving body based on correlation between the plurality of motion data items; and training an artificial neural network model using a training dataset including at least one motion data item among the plurality of motion data items as an input data item, and the coordination between the plurality of parts as a target variable.

In accordance with a second aspect of the present disclosure, there is provided an artificial neural network model training apparatus for inferring motion coordination, the apparatus comprises: a memory configured to store one or more programs; and a processor configured to execute the one or more stored programs, wherein the processor is configured to acquire a plurality of motion data items including each motion data item for a plurality of parts of a moving body; calculate coordination between parts of the moving body based on correlation between the plurality of motion data items; and train an artificial neural network model using a training dataset including at least one motion data item among the plurality of motion data items as an input data item, and the coordination between the plurality of parts as a target variable.

In accordance with a third aspect of the present disclosure, there is provided a motion coordination inferring method performed by a motion coordination inferring apparatus, the method comprises: preparing an artificial neural network model trained using a training dataset which comprises, as an input data item, at least one motion data for learning among a plurality of motion data items including motion data items for a plurality of parts of a moving body and, as a target variable, coordination between parts of the moving body calculated based on correlation between the plurality of motion data items; and inputting the measured motion data item obtained by the sensor to the trained artificial neural network model, and checking the coordination between parts of the target moving body, which is output by the trained artificial neural network model.

In accordance with a fourth aspect of the present disclosure, there is provided a motion coordination inferring apparatus, the apparatus comprises: a sensor configured to acquire a motion data item measured for at least one body part among a plurality of motion data items that correspond to motion data items for a plurality of parts of a target moving body; a memory configured to store one or more programs; and a processor configured to execute the one or more stored programs, wherein the processor comprises an artificial neural network model trained using a training dataset which comprises, as an input data item, at least one motion data for learning among a plurality of motion data items including motion data items for a plurality of parts of a moving body and, as a target variable, coordination between parts of the moving body calculated based on correlation between the plurality of motion data items, and wherein the processor is configured to input the measured motion data item obtained by the sensor to the trained artificial neural network model, and check the coordination between parts of the target moving body, which is output by the trained artificial neural network model.

In accordance with a fifth aspect of the present disclosure, there is provided a non-transitory computer-readable recording medium storing a computer program, which comprises instructions for a processor to perform an artificial neural network model training method.

In accordance with a sixth aspect of the present disclosure, there is provided a computer program stored in a non-transitory computer-readable recording medium, which comprises instructions for a processor to perform a motion coordination inferring method.

According to an embodiment of the present disclosure, in order to measure coordination between parts during movement of a moving body such as a human, it is possible to infer and measure the coordination between parts using only motion data items of some parts, without using the motion data items of all parts. For example, in order to measure coordination between parts of both arms in an activity such as walking, it is possible to infer and measure coordination between parts of the both arms using only motion data of one arm. That is, it is possible to reduce the number of sensors necessary to measure motion data items.

### DESCRIPTION OF DRAWINGS

FIG. 1 is a block diagram of an artificial neural network model training apparatus according to an embodiment of the present disclosure.

FIG. 2 is a flowchart illustrating an artificial neural network model training method performed by an artificial neural network model training apparatus according to an embodiment of the present disclosure.

FIG. 3 is a block diagram of a motion coordination inferring apparatus using an artificial neural network model according to an embodiment of the present disclosure.

FIG. 4 is a flowchart illustrating a motion coordination inferring method performed by an inferring motion coordination inferring apparatus using an artificial neural network model according to an embodiment of the present disclosure.

FIG. 5 is a diagram illustrating an example of a wearable sensor for measuring movement of both arms of a body according to an embodiment of the present disclosure.

FIGS. 6 and 7 are views illustrating motion data items measured by a wearable device including inertial sensors worn on wrists of both hands while a target moving body is walking.

FIGS. 8 and 9 are graphs for comparing values (y) obtained by measuring motion data items according to movement of a target moving body with respect to any one of the multiple parts of the target moving body and values (y_pred) obtained as an inferred motion data item for a corresponding part by a motion coordination inferring apparatus according to an embodiment of the present disclosure.

### MODE FOR DISCLOSURE

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

Terms used in the present specification will be briefly described, and the present disclosure will be described in detail.

In terms used in the present disclosure, general terms currently as widely used as possible while considering functions in the present disclosure are used. However, the terms may vary according to the intention or precedent of a technician working in the field, the emergence of new technologies, and the like. In addition, in certain cases, there are terms arbitrarily selected by the applicant, and in this case, the meaning of the terms will be described in detail in the description of the corresponding invention. Therefore, the terms used in the present disclosure should be defined based on the meaning of the terms and the overall contents of the present disclosure, not just the name of the terms.

When it is described that a part in the overall specification “includes” a certain component, this means that other components may be further included instead of excluding other components unless specifically stated to the contrary.

In addition, a term such as a “unit” or a “portion” used in the specification means a software component or a hardware component such as FPGA or ASIC, and the “unit” or the “portion” performs a certain role. However, the “unit” or the “portion” is not limited to software or hardware. The “portion” or the “unit” may be configured to be in an addressable storage medium, or may be configured to reproduce one or more processors. Thus, as an example, the “unit” or the “portion” includes components (such as software components, object-oriented software components, class components, and task components), processes, functions, properties, procedures, subroutines, segments of program code, drivers, firmware, microcode, circuits, data, database, data structures, tables, arrays, and variables. The functions provided in the components and “unit” may be combined into a smaller number of components and “units” or may be further divided into additional components and “units”.

Hereinafter, the embodiment of the present disclosure will be described in detail with reference to the accompanying drawings so that those of ordinary skill in the art may easily implement the present disclosure. In the drawings, portions not related to the description are omitted in order to clearly describe the present disclosure.

Hereinafter, the present invention will be described more fully with reference to the accompanying drawings for one of ordinary skill in the art to be able to perform the present invention without any difficulty. Also, parts in the drawings unrelated to the detailed description are omitted to ensure clarity of the present invention. In the following description, as long as a moving body subject to motion coordination inference includes a plurality of moving parts having organic motion characteristics, all animals as well as humans may be a target.

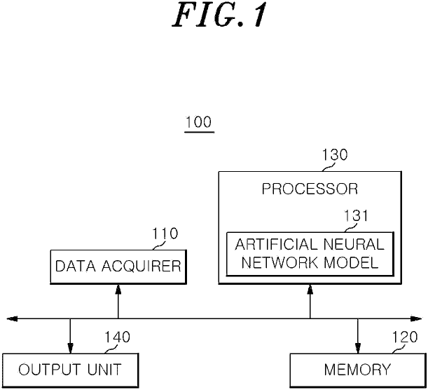

FIG. 1 is a block diagram of an artificial neural network model training apparatus 100 according to an embodiment of the present disclosure.

According to an embodiment, the artificial neural network model training apparatus 100 includes a data acquirer 110, a memory 120, and a processor 130, and may further include an output unit 140.

The data acquirer 110 acquires a plurality of motion data items corresponding to respective motion data items for a plurality of parts according to movement of a moving body, and provides the acquired plurality of motion data items to the memory 120 and/or the processor 130. For example, the data acquirer 110 may include a sensor for measuring motions of two or more parts of a plurality of parts of the moving body, and in this case, a motion data item measured by measuring a motion of each part of the moving body may be provided to the memory 120 and/or the processor 130. Alternatively, sensors for measuring movement of two or more parts among a plurality of parts of the moving body may be provided separately from the data acquirer 110, and the data acquirer 110 may be provided with a plurality of measured motion data items measured by the sensors provided separately. For example, the data acquirer 110 may receive a plurality of motion data items measured by the separately provided sensors through an input interface. Alternatively, the data acquirer 110 may receive a plurality of motion data items measured by the separately provided sensors through a communication channel.

The memory 120 stores one or more programs. In the memory 120, a computer program for controlling the processor 130 of the artificial neural network model learning device 100 to perform an artificial neural network model training method may be stored, and results of various processing by the processor 130 may be stored.

The processor 130 may execute one or more programs stored in the memory 120. The processor 130 calculates coordination between parts of the moving body based on a similarity between the plurality of motion data items of the learning exercise body, and trains an artificial neural network model 131 by using a learning dataset including at least one motion data item among a plurality of motion data items as an input and the calculated coordination between parts as a target variable. For example, the plurality of motion data items may be motion data items for a plurality of moving parts having organic motion characteristics, and a balance scoring result value for the plurality of moving parts may be calculated as the coordination between parts of the moving body. For example, the motion data items may be measured by inertial sensors mounted on two or more of the plurality of moving parts. Here, when calculating the coordination between parts, the calculation may be implemented using a cross-correlation value or using a general algorithm for identifying similarity between data items, such as dynamic time warp analysis.

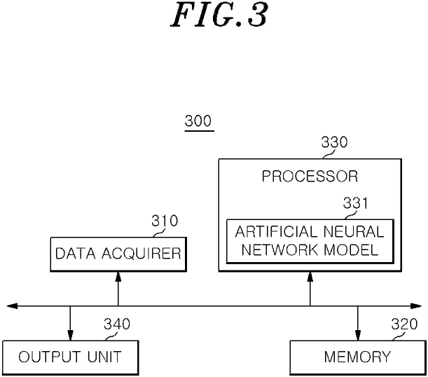

The output unit 140 may output various results of processing by the processor 130. For example, the output unit 140 may output various results of processing by the processor 130 in a written form or a screen form so as to be seen outside. Alternatively, the output unit 140 may transmit the various results of processing by the processor 130 to a peripheral device through an output interface or to another device (e.g., a motion coordination inferring apparatus of FIG. 3) through a communication channel. The output unit 140 transmits the artificial neural network model 131 trained using a training dataset, which includes at least one motion data item among a plurality of motion data items as an input and the calculated coordination between parts as a target variable, to the motion coordination inferring apparatus of FIG. 3.

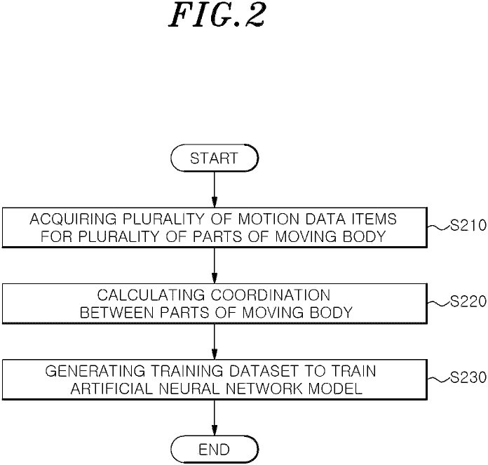

FIG. 2 is a flowchart illustrating an artificial neural network model training method performed by the artificial neural network model training apparatus 100 according to an embodiment of the present disclosure. The artificial neural network model training method will be described below.

FIG. 3 is a block diagram of a motion coordination inferring apparatus 300 using an artificial neural network model according to an embodiment of the present disclosure.

According to an embodiment, the motion coordination inferring apparatus 300 includes a data acquirer 310, a memory 320, and a processor 330, and may further include an output unit 340.

The data acquirer 310 acquires a motion data item measured for a part corresponding to at least one motion data item among a plurality of motion data items corresponding to respective motion data items for a plurality of parts according to movement of a target moving body, and provides the acquired plurality of motion data items to the memory 320 and/or the processor 330. For example, the data acquirer 310 may include a sensor for measuring respective motions of two or more parts of a plurality of parts of the target moving body, and in this case, a motion data item measured by measuring a motion of each part of the target moving body may be provided to the memory 120 and/or the processor 130. Alternatively, sensors for measuring movement of two or more parts among a plurality of parts of the target moving body may be provided separately from the data acquirer 310, and the data acquirer 310 may be provided with a plurality of measured motion data items measured by the sensors provided separately. For example, the data acquirer 210 may receive a plurality of motion data items measured by the separately provided sensors through an input interface. Alternatively, the data acquirer 310 may receive a plurality of motion data items measured by the separately provided sensors through a communication channel.

The memory 320 stores one or more programs. In the memory 320, a computer program for controlling the processor 330 of the motion coordination inferring apparatus 300 to perform a motion coordination inferring method may be stored in the memory 320 and various results of processing by the processor 330 may be stored.

The processor 330 may execute one or more programs stored in the memory 320. The processor 330 includes the artificial neural network 331, which has learned a training dataset that includes, as an input, at least one motion data item for learning among a plurality of motion data items corresponding to motion data items for a plurality of parts according to movement of the moving body and, as a target variable, the coordination between parts of the moving body calculated based on the similarity between the plurality of motion data items. Then, the processor 330 inputs the motion data items of the target moving body, which are provided to the data acquirer 310, to the pre-trained artificial neural network model 331, and infer the coordination between parts of the target moving body by using an output from the pre-trained artificial neural network model 331. In the case of body movements, movements between body parts have a specific correlation. For example, in walking and running activities, a rhythmic and symmetrical pattern of movements of the left and right hands is observed. This is due to biological neural circuits that generate rhythmic outputs of a body, which are called Central Pattern Generators (CPGs), and healthy people can move organically with regular relationships between body parts without much effort. By using the principle of the characteristics of coupled movements between body movements, it is possible to measure a similarity with a value of a sensor whose measurement is omitted, without attaching sensors to all target parts to be measured.

Then, the processor 330 may generate information representing motion characteristics of the target moving body based on the inferred coordination between parts of the target moving body, and may control the output unit 340 to output the generated information representing the motion characteristics of the target moving body. For example, the plurality of motion data items may be motion data items for a plurality of moving parts having organic motion characteristics and a balance scoring result value for the plurality of moving parts may be used as coordination between parts of a moving body and/or target moving body. For example, the motion data items may be measured by inertial sensors mounted on two or more of the plurality of moving parts.

The output unit 340 may output various results of processing by the processor 330. For example, the output unit 340 may output the various results of processing by the processor 330 in a written form or a screen form so as to be seen outside. Alternatively, the output unit 340 may transmit the various results of processing by the processor 330 to a peripheral device through an output interface or to another device through a communication channel. For example, the output unit 340 may output information representing motion characteristics of the target moving body under the control of the processor 330.

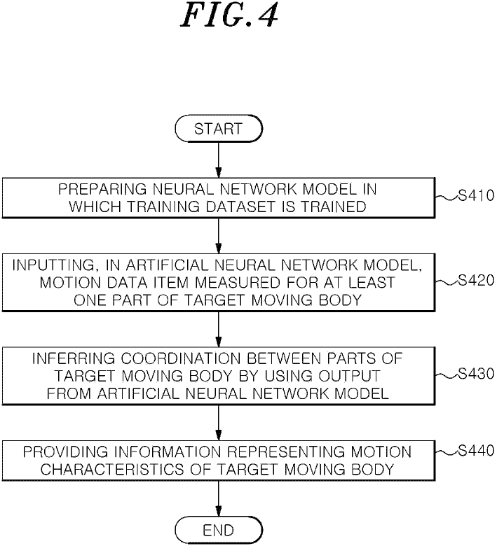

FIG. 4 is a flowchart illustrating a method of inferring motion coordination using an artificial neural network model, the method performed by the motion coordination estimation apparatus 300 according to an embodiment of the present disclosure. The method for inferring motion coordination will be described below.

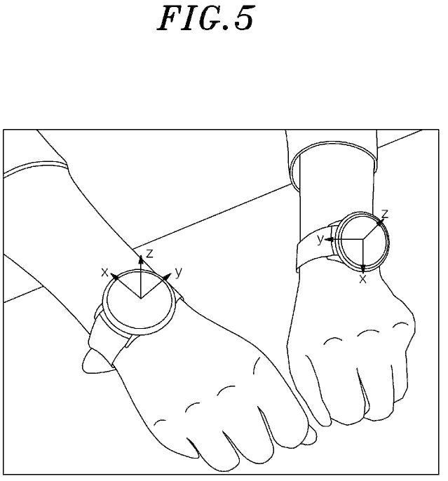

FIG. 5 is a diagram illustrating an example of a sensor that can be used by the artificial neural network model training apparatus 100 according to an embodiment of the present disclosure to acquire a motion data item for each of a plurality of parts according to movement of a moving body or to acquire a motion data item for each of a plurality of parts of the target moving body. For example, wearable devices having inertial sensors therein may be worn on the wrists of both hands to detect motion data items of the both hands according to movement of a human body.

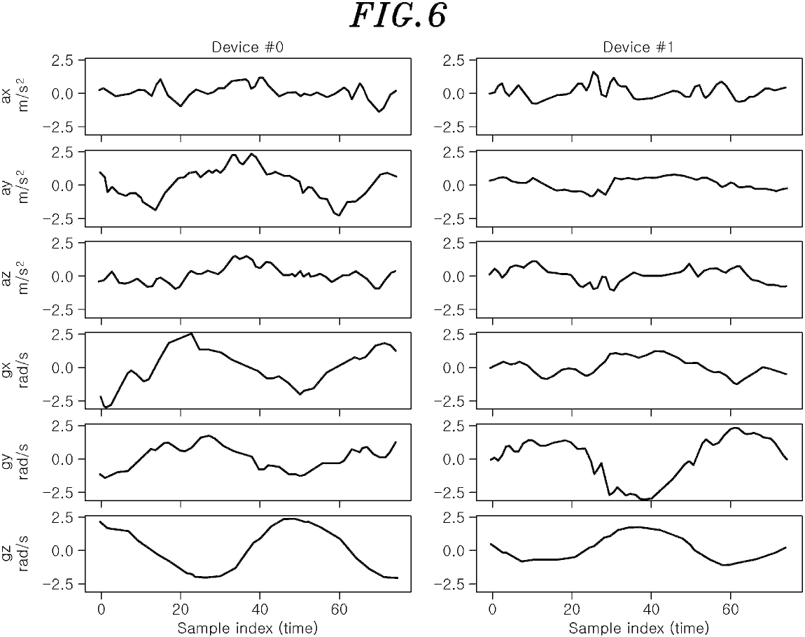

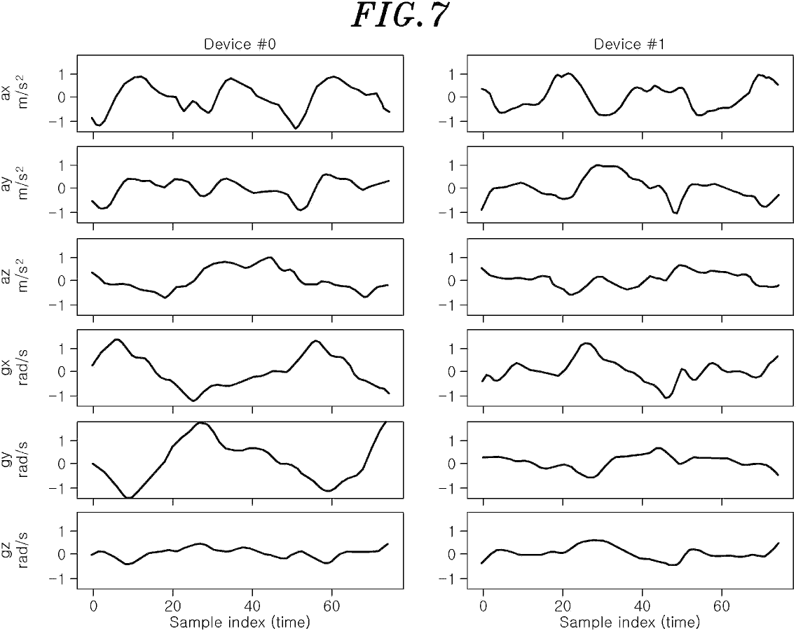

FIGS. 6 and 7 are views illustrating motion data items measured by a wearable device including inertial sensors worn on wrists of both hands while a target moving body is walking.

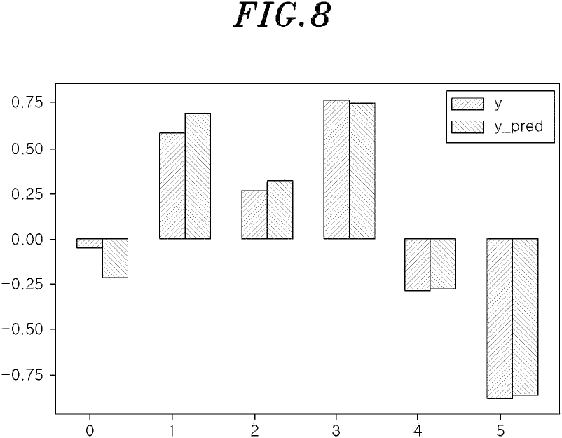

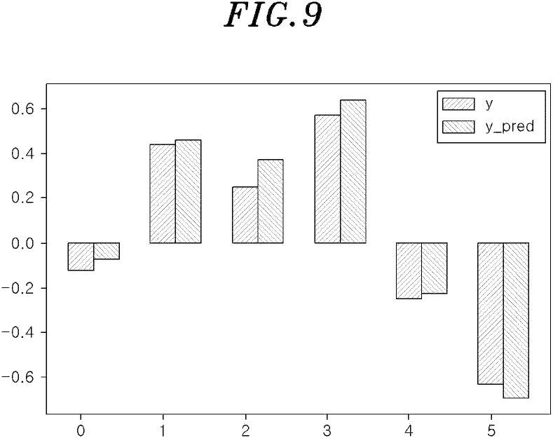

FIGS. 8 and 9 are graphs for comparing values (y) obtained by measuring motion data items according to movement of a target moving body with respect to any one of the plurality of parts of the target moving body and values (y_pred) obtained as an inferred motion data item for a corresponding part by the motion coordination inferring apparatus 200 according to an embodiment of the present invention.

As described above with reference to the accompanying drawings, the artificial neural network model training apparatus 100 and the motion coordination inferring apparatus 300 may be implemented as separate devices, but may be implemented as a single device depending on embodiments. For example, the artificial neural network model training apparatus 100 and the motion coordination inferring apparatus 300 may be the same device capable of performing all the functions, and the same device capable of performing all the functions may perform the artificial neural network model training method as shown in FIG. 2 and the motion coordination inferring method using the artificial neural network model as shown in FIG. 4.

Hereinafter, an artificial neural network model training method performed by the artificial neural network model training apparatus 100 and a motion coordination inferring method performed by the motion coordination inferring apparatus 300 according to an embodiment of the present disclosure will be described in detail. An embodiment will be described, in which a learning dataset based on motion data items sensed while the wearable devices including inertial sensors, as illustrated in FIG. 5, are worn on the wrists of both hands is learned and a motion data item corresponding to any one hand among pre-learned training dataset among a motion data item corresponding to one hand of the target moving body is input to an artificial neural network model to infer coordination between the two hands of a target moving body by using an output from the artificial neural network model. That is, the plurality of moving parts are both hands among the limbs, and an example of using a plurality of motion data items measured for the both hands as a plurality of moving parts having organic motion characteristics will be described.

First, the data acquirer 110 of the artificial neural network model training apparatus 100 acquires motion data items of both hands as each motion data items for a plurality of parts according to movement of the moving body, and provides the acquired motion data items to the processor 130. For example, the data acquirer 110 may include a receiver capable of receiving data through a communication channel, and may receive motion data items, as shown in FIG. 6, sensed in both hands by wearable devices having inertial sensors shown in FIG. 6, and may provide the received motion data items to the processor 130. For example, Device #0 providing the motion data items of FIG. 6 may be a wearable device worn on the left hand shown in FIG. 5, and Device #1 may be a wearable device worn on the right hand illustrated in FIG. 5. In FIG. 6, az, ay, and ax are accelerations, and gz, gy, and gx are angular velocities (S210).

Then, the processor 130 of the artificial neural network model training apparatus 100 calculates coordination between parts of the moving body based on similarity between a plurality of motion data items of the moving body. For example, with respect to motion data items for a plurality of moving parts having organic motion characteristics, the processor 130 may calculate a balance scoring result value for the plurality of moving parts as coordination between parts of a moving body for learning. For example, the processor 130 may calculate coordination between parts of a hand by using a cross-correlation value for motion data items of both hands, which is provided by the data acquirer 110, or a general algorithm for identifying similarity between data items, such as dynamic time warp analysis (S220).

Then, the processor 130 includes a dataset, which includes at least one motion data item among a plurality of motion data items, for example, a motion data item of a left hand or a motion data item of a right hand, as an input and the coordination between parts, which is calculated in operation S210, as a target variable, and trains the artificial neural network model 131 using the generated training dataset (S230).

And, the processor 130 may control the output unit 140 of the artificial neural network model training apparatus 100 to output the artificial neural network model 131 trained in operation S230, and the output unit 141 may output the pre-trained artificial neural network model 131 under the control of the processor 130. For example, the output unit 140 may transmit the pre-trained artificial neural network model 131 to the motion coordination inferring apparatus 300 through a communication channel. Accordingly, the motion coordination inferring apparatus 300 may be able to infer coordination between parts of the target moving body using the artificial neural network model.

Meanwhile, the data acquirer 310 of the motion coordination inferring apparatus 300 may receive the pre-trained artificial neural network model from the artificial neural network model training apparatus 100 and provide the received pre-trained artificial neural network model to the processor 330 of the motion coordination inferring apparatus 300. This means that the artificial neural network model 331 is prepared by the motion coordination inferring apparatus 300 and thus the processor 330 is ready to infer coordination between parts of the target moving body using the artificial neural network model (S420).

Then, the data acquirer 310 acquires a motion data item measured for a part corresponding to at least one motion data item among a plurality of motion data items corresponding to respective motion data items for a plurality of parts according to movement of a target moving body, and provides the acquired plurality of motion data items to the processor 330. For example, the data acquirer 310 may include a receiver capable of receiving data through a communication channel, and may receive motion data items, as shown in FIG. 6, sensed in both hands by wearable devices having inertial sensors shown in FIG. 6, and may provide the received motion data items to the processor 330. For example, in the learning process of operation S230, when a motion data item of a left hand is included in the training dataset, the data acquirer 310 may provide the processor 130 with a motion data item received from a wearable device worn on the left hand of the target moving body.

Then, the processor 330 inputs the motion data item of the target moving body provided by the data acquirer 310 to the pre-trained artificial neural network model 331 (S420), and infers coordination between parts of the target moving body by using an output of the pre-learned artificial neural network model 331 (S430).

Then, the processor 330 may generate information representing the motion characteristics of the target moving body based on the coordination between parts of the target moving body inferred in operation S430, and may control the output unit 340 to output the generated information representing the motion characteristics of the target moving body. Then, the output unit 340 may output the information representing the motion characteristics of the target moving body under the control of the processor 330.

Here, the processor 330 may process and output the coordination between parts of the target moving body inferred in operation S430 in a form outputtable through the output unit 340, or may provide an explanatory text for motion characteristics of the target moving body based on the coordination between parts of the target moving body inferred in operation S430. For example, regarding motion data items shown in FIG. 7, when comparing the gy graphs of both hands, it can be seen that there is a large difference in the characteristics of the high and low changes. In this case, the processor 330 may generate an explanatory text indicating that one hand of the target moving body has more motion characteristics compared to the other hand, and output the generated explanatory text through the output unit 340. Alternatively, when comparing the gz graph of both hands shown in FIG. 7 and the gz graph of both hands shown in FIG. 6, it can be seen that the gz graph of FIG. 7 has a lower amplitude than the gz graph of FIG. 6. In this case, the processor 330 may infer that the target moving body has a gait disorder, which has influenced the movement of the hand having organic motion characteristics, and may generate an explanatory text indicating that the target moving body has a gait disorder characteristic and output the explanatory text through the output unit 340 (S440).

FIGS. 8 and 9 are graphs for comparing values (y) obtained by measuring motion data items according to movement of a target moving body with respect to any one of the plurality of parts of the target moving body and values (y_pred) obtained as inferred motion data item for a corresponding part by the motion coordination inferring apparatus 200 according to an embodiment of the present invention. As can be seen from FIGS. 8 and 9, in most cases, there is no significant difference between the inferred values (y_pred) and the actually measured values (y).

As described so far, according to an embodiment of the present disclosure, in order to measure coordination between parts during movement of a moving body such as a human, it is possible to infer and measure coordination between parts using only motion data items of some parts without using motion data items of all parts. For example, in order to measure coordination between parts of both arms in an activity such as walking, it is possible to infer and measure coordination between parts of the both arms using only motion data of one arm. That is, it is possible to reduce the number of sensors necessary to measure motion data items.

Meanwhile, the respective steps included in the artificial neural network model training method for inferring motion coordination and the motion coordination inferring method according to the above-described embodiment may be implemented in a computer readable recording medium having recorded thereon a computer program including instructions for performing these steps.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

1] An artificial neural network model training method performed by an artificial neural network model training apparatus for inferring motion coordination, the method comprising:
acquiring a plurality of motion data items including each motion data item for a plurality of parts of a moving body;
calculating coordination between parts of the moving body based on correlation between the plurality of motion data items; and
training an artificial neural network model using a training dataset including at least one motion data item among the plurality of motion data items as an input data item, and the coordination between the plurality of parts as a target variable.

2] The artificial neural network model training method of claim 1,
wherein the plurality of motion data items includes motion data items for a plurality of moving parts having organic motion characteristics among the plurality of parts of the moving body, and
wherein the calculating of the coordination between the plurality of parts of the moving body includes calculating a balance scoring result for the plurality of moving parts as the coordination between the plurality of parts.

3] The artificial neural network model training method of claim 2, wherein the motion data item includes a data item measured by inertial sensors mounted on two or more of the plurality of moving parts.

4] The artificial neural network model training method of claim 1, wherein the calculating of the coordination between the plurality of parts of the moving body includes determining the correlation between the data items using a cross correlation value or dynamic time warp analysis.

5] A motion coordination inferring apparatus comprising:
a sensor configured to acquire a motion data item measured for at least one body part among a plurality of motion data items that correspond to motion data items for a plurality of parts of a target moving body;
a memory configured to store one or more programs; and
a processor configured to execute the one or more stored programs,
wherein the processor comprises an artificial neural network model trained using a training dataset which comprises, as an input data item, at least one motion data for learning among a plurality of motion data items including motion data items for a plurality of parts of a moving body and, as a target variable, coordination between parts of the moving body calculated based on correlation between the plurality of motion data items, and
wherein the processor is configured to input the measured motion data item obtained by the sensor to the trained artificial neural network model, and check the coordination between parts of the target moving body, which is output by the trained artificial neural network model.

6] The motion coordination inferring apparatus of claim 5, further comprising:
an output unit configured to output a result of processing by the processor,
wherein the processor is configured to generate information representing motion characteristics of the target moving body based on the inferred coordination between the plurality of parts of the target moving body, and
wherein the output unit is configured to output information representing motion characteristics of the target moving body under control of the processor.

7] The motion coordination inferring apparatus of claim 5, wherein the plurality of motion data items includes motion data items for a plurality of moving parts having organic motion characteristics of the learning motion body or the target moving body, and
wherein the balance scoring result for the plurality of moving parts is used as the coordination between the parts.

8] The motion coordination inferring apparatus of claim 7, wherein the motion data item is measured by an inertial sensor mounted on at least one moving part among the plurality of moving parts.

9] A computer-readable recording medium storing a computer program thereon, the medium comprising instructions for controlling a processor to perform an artificial neural network model training method for inferring motion coordination,
wherein the computer program, when executed by the processor, performs the following operations:
acquiring a plurality of motion data items including motion data items for a plurality of parts of a moving body;
calculating coordination between the parts of the moving body based on correlation between the plurality of motion data items; and
training an artificial neural network model using a training dataset including at least one motion data item among the plurality of motion data items as an input data item and the coordination between the parts as a target variable.

10] The computer-readable recording medium of claim 9,wherein the plurality of motion data items includes motion data items for a plurality of moving parts having organic motion characteristics among the plurality of parts of the moving body, and
wherein the calculating of the coordination between the plurality of parts of the moving body includes calculating a balance scoring result for the plurality of moving parts as the coordination between the plurality of parts.

11] The computer-readable recording medium of claim 10, wherein the motion data items are data items measured by inertial sensors mounted on two or more of the plurality of moving parts.

12] The computer-readable recording medium of claim 9, wherein the calculating of the coordination between the plurality of parts of the moving body includes determining the correlation between the data items using a cross correlation value or dynamic time warp analysis.
